(19) United States
(12) Patent Application Publication
(10) Pub. No.: US 2022/0314215 A1
(43) Pub. Date: Oct. 6, 2022

(54) VERTICALLY LAYERED FLUID COLUMNS

(71) Applicant: Hewlett-Packard Development Company, L.P., Spring, TX (US)

(72) Inventors: Hilary ELY, Corvallis, OR (US); Si-lam J. CHOY, Corvallis, OR (US)

(73) Assignee: Hewlett-Packard Development Company, L.P., Spring, TX (US)

(21) Appl. No.: 17/642,265

(22) PCT Filed: May 8, 2020
(86) PCT No.: PCT/US2020/032187

§ 371 Date: Mar. 11, 2022

Related U.S. Application Data
(63) Continuation of application No. PCT/US2019/058429, filed on Oct. 29, 2019.
Application No. PCT/US2019/058429 is a continuation of application No. PCT/US2019/058427, filed on Oct. 29, 2019.

(51) Int. Cl. B01L 3/00; G01N 35/00; C12N 15/10

(52) U.S. Cl. CPC B01L 3/5088; G01N 35/0098; C12N 15/1013; B01L 2200/0631

(57) ABSTRACT

A vertically layered fluid column can include a plurality of fluids positioned in fluid layers, a density-differential interface along which two fluids from the plurality of fluids are positioned, and a capillary force-supported interface along which two fluids from the plurality of the fluids are positioned.

### BACKGROUND

In biomedical, chemical, and environmental testing, isolating a component of interest from a sample fluid can be useful. Such separations can permit analysis or amplification of a component of interest. As the quantity of available assays for components increases, so does the demand for the ability to isolate components of interest from sample fluids.

### BRIEF DESCRIPTION OF THE DRAWING

FIG. 1 graphically illustrates a schematic view of an example vertically layered fluid column in accordance with the present disclosure;

FIG. 2 graphically illustrates an alternative schematic view of a vertically layered fluid column in accordance with the present disclosure;

FIG. 3 graphically illustrates a schematic view of an example biological component processing system with particulate substrates pre-loaded in a first fluid to move through a vertically layered fluid column in accordance with the present disclosure;

FIG. 4 graphically illustrates a schematic view of an alternative example biological component processing system with particulate substrates included as a kit to load into and move through a vertically layered fluid column using a magnetic field in accordance with the present disclosure;

FIG. 5 graphically illustrates a schematic view of an example biological component processing system including multiple vertically layered fluid columns with two multi-fluid density gradient portions and separated by a capillary force gradient portion, which can be connected together fluidically in series in accordance with examples of the present disclosure;

FIG. 6 is a flow diagram illustrating an example method of processing a biological component from a biological sample in accordance with examples of the present disclosure; and

FIG. 7 graphically illustrates a more specific example method of processing includes concentrating a nucleic acid from a biological sample in accordance with the present disclosure.

### DETAILED DESCRIPTION

In biological assays, a biological component can be intermixed with other components in a biological sample that can interfere with subsequent analysis. As used herein, the term “biological component” can refer to materials of various types, including proteins, cells, cell nuclei, nucleic acids, bacteria, viruses, or the like, that can be present in a biological sample. A “biological sample” can refer to a fluid or a dried or lyophilized material obtained for analysis from a living or deceased organism. Isolating the biological component from other components of the biological sample can permit subsequent analysis without interference and can increase an accuracy of the subsequent analysis. In addition, isolating a biological component from other components in a biological sample can permit analysis of the biological component that would not be possible if the biological component remained in the biological sample. Many of the current isolation techniques can include repeatedly dispersing and re-aggregating samples. The repeated dispersing and re-aggregating can result in a loss of a quantity of the biological component. Furthermore, isolating a biological component with some of these techniques can be complex, time consuming, and labor intensive and can also result in less than maximum yields of the isolated biological component.

In accordance with examples of the present disclosure, a vertically layered fluid column includes three fluids positioned in fluid layers. Two of the fluids are positioned along a density-differential interface and two of the fluids are positioned along a capillary force-supported interface. In one example, one of the fluids can be positioned along the density-differential interface and can also be positioned along the capillary force-supported interface, e.g., three fluid fluids positioned as layers with a middle layer including two interfaces (one along an upper side and one along a bottom side of the fluid layer). In another example, one of the fluids can be an oil and is positioned along a capillary force-supported interface. In another example, vertically layered fluid column may include four fluids positioned as fluid layers. In this example, two of the fluids can be positioned along the density-differential interface and the two of the fluids can be positioned along the capillary force-supported interface. In further detail, one of the fluids can be a gas, e.g., air, and one of the fluids can be an oil, e.g., mineral oil, and the gas and the oil can be positioned along the capillary force-supported interface. In another example, a biological sample can be included having a biological component that can pass through the density-differential interface and the capillary force-supported interface. In one example, the capillary force-supported interface can separate a lower fluid from an upper fluid, wherein the lower fluid is less dense than the upper fluid. The capillary force-supported interface can be, for example, contained within a fluidic channel having a cross-sectional dimension that is aligned with the capillary force-supported interface is less than 2 mm.

In another example, a biological component processing system includes a vertically layered fluid column including three fluids positioned in fluid layers, wherein two of the fluids are positioned along a density-differential interface, and wherein two of the fluids are positioned along a capillary force-supported interface. The biological component processing system also includes a particulate substrate to pass through the density-differential interface and the capillary force-supported interface. The particulate substrate can include magnetizing particles, for example. In this example, the vertically layered fluid column can be spatially positioned adjacent to a magnet to provide a magnetic field, e.g., to move the magnetizing particles along a z-axis through the density-differential interface and through the capillary force-supported interface. In another example, the particulate substrate can include microparticles that have a density greater than the three fluids positioned in fluid layers, wherein the density of the microparticles is sufficient to allow the microparticles to pass through the density-differential interface and the capillary force-supported interface either by the force of gravity or centrifugation. The particulate substrate can include particle surfaces that are associated with a biological component. A biological sample can also be included, where the particulate substrate includes particle surfaces that are surface-activated to preferentially bind with a biological component relative to secondary components in the biological fluid sample.

In another example, a method of processing a biological component from a biological sample includes loading a biological sample into a vertically layered fluid column including a plurality of fluids positioned in fluid layers, a density-differential interface along which two fluids from the plurality of fluids are positioned, and a capillary force-supported interface along which two fluids from the plurality of the fluids are positioned. The method in this example also includes passing a biological component of the biological sample through the density-differential interface and the capillary force-supported interface.

It is noted that when discussing examples of vertically layered fluid columns, biological component processing systems, or methods of processing a biological component from a biological sample, such discussions can be considered applicable to one another whether or not they are explicitly discussed in the context of that example. Thus, for example, when discussing a multi-fluid density gradient portion in a vertically layered fluid column, such disclosure is also relevant to and directly supported in the context of a microfluidic biological component processing system, or a method of processing a biological component from a biological sample, and vice versa.

Terms used herein will have the ordinary meaning in the relevant technical field unless specified otherwise. In some instances, there are terms defined more specifically throughout the specification or included at the end of the present specification, and thus, these terms can have a meaning as described herein.

### Vertically Layered Fluid Columns

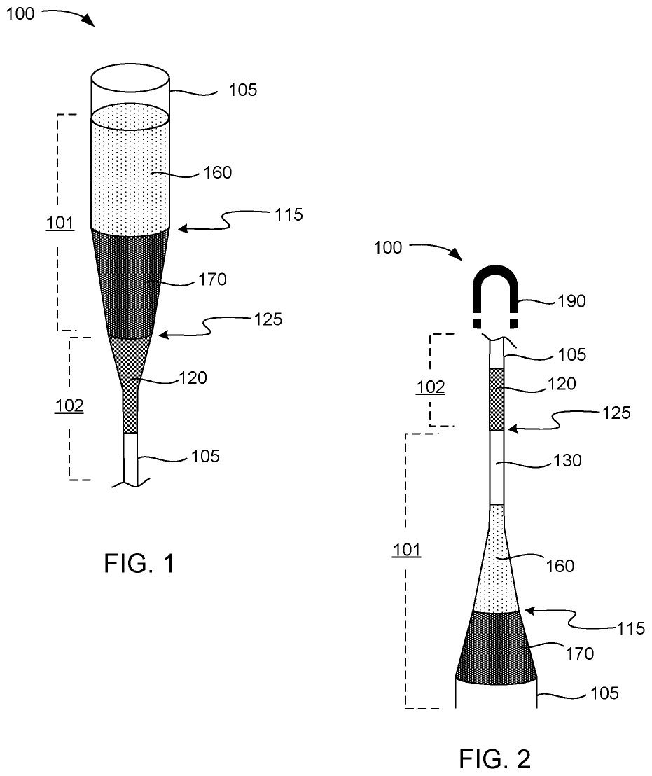

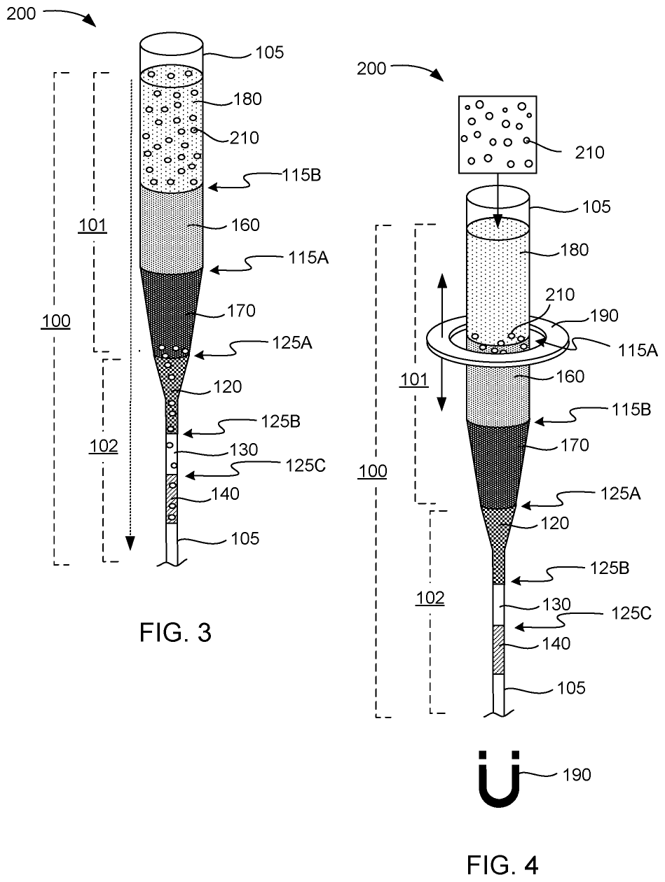

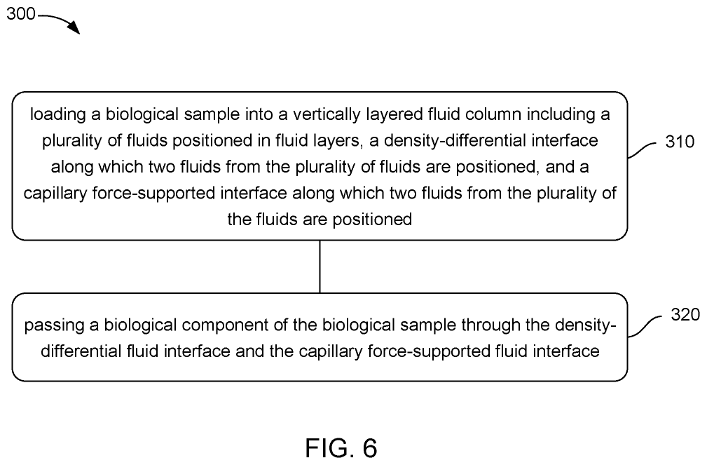

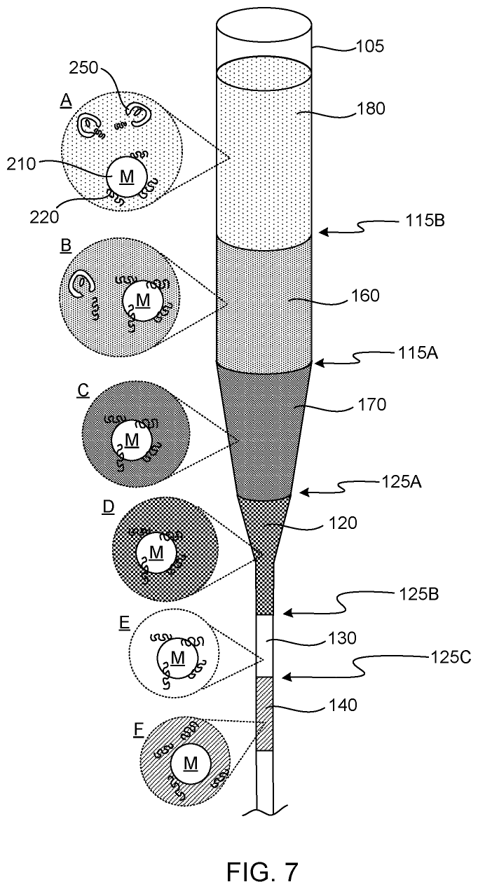

FIGS. 1 and 2 illustrate two different example vertically layered fluid columns. These vertically layered fluid columns can be used or constructed for the biological component processing systems shown and described hereinafter by way of example in FIGS. 3-5 and/or in the context of the methods of processing biological components from biological fluid samples shown and described hereinafter by way of example in FIGS. 6-7.

With respect more specifically to the vertically layered fluid columns of the present disclosure, as shown in FIG. 1, a specific vertically layered fluid column 100 can include two portions, namely a multi-fluid density gradient portion 101 and a capillary force gradient portion 102. The two portions may be contained or supported by a vessel 105, which can include both an enlarged portion positioned about the vertically layered fluid column along the multi-fluid density gradient portion (where capillary forces are not used to provide the density-differential interface 115), and a narrower portion which may include a capillary tube positioned about the vertically layered fluid column along the capillary force gradient portion (where capillary forces do contribute to forming the capillary force-supported interface 125).

As an initial matter, the terms “density gradient” or “multi-fluid density gradient” can be used in various contexts herein but refer to the ability of multiple fluids to remain separated in layers due to their density difference (with denser fluids being positioned vertically lower along the column). Thus, there can be multiple fluids that are phase separated, but are still in direct contact at a fluid interface, referred to herein as a “density-differential interface,” which is descriptive of the interface being present as a result of the density difference.

The terms “capillary force” or “capillary force-supported gradient” refer to fluid interfaces that are not provided by their increasing density and their density difference, but rather, the fluids of immediately adjacent layers can have different densities, but less dense fluids can be positioned below denser fluids, and the reason these less dense fluid do not migrate upward is because they are constrained within a fluidic channel due to the surface tension of the fluids at the fluid interface and the interaction of the fluids with walls of this channel portion of the vessel, namely at the “capillary force-supported interface.”

Furthermore, in referring to the figures, as there are several fluids often being described, they may be referred to as a “first,” “second,” “third,” etc., fluid so that they can be described relative to one another and for clarity in describing for understanding the disclosure, but should not be considered to be limiting. For example, fluid 180 could be referred to as the “first fluid,” fluid 170 could be referred to as the “second fluid,” and fluid 160 could be referred to as the “third fluid” or the “fourth fluid,” etc., without consequence to the scope of the disclosure. The mentioning of “first,” second,” “third,” etc., should be viewed in the context of the other layers in the immediately described vertically layered fluid column, biological component processing system, or method of process a biological component, and not confused with other instances where the terms “first,” “second,” “third,” etc., may be used differently in another context. For example, when there are more than three or so fluids, and when there are multiple types of the various fluid interfaces, it may make sense to dispense with the use of “first,” “second,” “third,” etc., with the understanding that these naming conventions can be assigned inferentially based on the spatial relationships from layer to layer.

Referring again to FIG. 1, the multi-fluid density gradient portion 101 includes two (or more) individual fluids of different densities. In this example, there is a first fluid 160 having a first fluid density and a second fluid 170 having a second fluid density that is greater than the first fluid density. The density difference between the first and the second fluid is sufficient so that the fluids remain phase separated at a (first) density-differential interface 115, even though the fluids are in direct contact with one another, e.g., the fluids are separated by their densities, not by a membrane or other artificial structure therebetween.

Example density differences of the first fluid relative to the second fluid (or any two fluids along the multi-fluid density gradient portion) can be from 50 mg/mL to 3 g/mL, from 100 mg/mL to 3 g/mL, from 500 mg/mL to 3 g/mL or from 1 g/mL to 3 g/mL The “fluid density” can be measured conventionally by calibrating a scale to zero with the container thereon and then obtaining the mass of the fluid, e.g., liquid, in grams. The volume of the measure mass can then be determined using a graduated cylinder. The density is then calculated by dividing the mass by the volume to get the fluid density (g/mL).

The vertically layered fluid column 100 also includes a third fluid 120 in this example. However, in this instance, the density of the third fluid is less dense than the second fluid 170. Thus, in a more standard sized column, the third fluid may otherwise migrate up into or through the second fluid, destroying the interface between the second and third fluids. However, in the example shown, this is not the case. The third fluid is constrained by the cross-sectional size of the vessel that contains the vertically layered fluid column (along the plane where the second fluid interfaces with the third fluid). Thus, the surface tension of the third fluid combined with the size constraint of the column at this interface in combination provide capillary force-supported interface 125, which promotes the second fluid and the third fluid remaining separated from one another. More specifically, the capillary force-supported interface can be contained within a fluidic channel having a cross-sectional dimension that is aligned with the capillary force-supported interface, and can be, for example, less than 2 mm. This dimension can be a diameter dimension for circular channels, or for non-circular geometries, this dimension can be the average cross-sectional dimension or the distance between opposing parallel surfaces of the channel, for instance. If the channel at this location is conical, the distance along the capillary force-supported interface can be used.

The first fluid 160, the second fluid 170, and the third fluid 120 can be any of a number of combinations of fluids, such as gas fluid, e.g., air, aqueous fluid, non-polar fluid, master mix fluid, reagent fluid, surfacing binding fluid, washing fluid, elution fluid, lysis fluid, etc. These or other fluids may be selected for use, and furthermore, these or other fluids may likewise be selected for use either above the first fluid or beneath the third fluid, as shown in FIG. 1.

Referring now to FIG. 2, an alternative vertically layered fluid column 100 is shown that can include two portions, namely a multi-fluid density gradient portion 101 and a capillary force gradient portion 102. The two portions may be contained or supported by a vessel 105, and can interact with the vessel (or not interact with the vessel) as previously described in FIG. 1. In FIG. 2, there is a first fluid 160 having a first fluid density and a second fluid 170 having a second fluid density that is greater than the first fluid density. The density difference between the first and the second fluid is sufficient so that the fluids remain phase separated at a (first) density-differential interface 115, even though the fluids are in direct contact with one another, e.g., the fluids are separated by their densities, not by a membrane or other artificial structure there between. The vertically layered fluid column also includes a third fluid 120 in this example. However, in this instance, the density of the third fluid is denser than a fourth fluid 130 that is positioned just beneath the third fluid. Because the third fluid and/or the fourth fluid is/are constrained by the cross-sectional size of the vertically layered fluid column (along the plane where the third fluid interfaces with the fourth fluid) and the surface tension of the fluid and/or the fourth fluid interacts with the vessel at that location that contains the vertically layered fluid column via capillary forces, the surface tension combined with the size constraint of the column (limited in cross-sectional size at the capillary force-supported interface) to provide the forces that allows for the formation of the capillary force-supported interface 125. This can be the case when a lower-positioned fluid (as defined by gravity or centripetal force acting on the column) is less dense than an upper-positioned fluid, or when the two fluids don't have enough density different to keep them otherwise separated. The dimensions, fluid details, and the like can be the same as described with reference to FIG. 1. In one specific example, the fourth fluid may be a gas, such as air, and the third fluid above may be master mix fluid or elution buffer or the like. In further detail, the third fluid may be a fluid that facilitates particulate substrates from passing from liquid (first fluid 160) to gas (fourth fluid 130), e.g., oil or other lubricant-type fluid. The particulate substrates can be drawn magnetically upward in a positive z-axis direction magnetically or by buoyance, for example, as described in greater detail hereinafter using a magnet 190, for example.

In further detail regarding the multi-fluid density gradient portion 101 of the vertically layered fluid column 100, there can be any of a number of fluids in this portion of the column, e.g., two fluids, three fluids, four fluids, etc., vertically arranged. Thus, a “multi-fluid density gradient portion” as used herein, can refer to a multi-layered fluid arranged with density gradient interfaces extending horizontally there between. The fluids may or may not be positioned 90 degrees from horizontal relative to one another, e.g., they may or may not be stacked or layered directly on top of one another but may be in a vessel angled at less than 90 degrees from horizontal, but the interface between the fluids are essentially horizontal. Thus, the term “vertically layered” refers to fluids that are on top of one another relative to a force such as gravity or centripetal force in a centrifuge with a horizontal interface extending there between, even if they are not fully directly on top of one another. A multi-fluid density gradient portion of the vertically layered fluid column does not include fluid layers where an additional substance may be used to separate one fluid layer from another. Fluid layers of the multi-fluid density gradient portion can be phase separated from one another based on fluidic properties of the various fluids, including density of the respective fluids along the column. The greater or higher the density of a fluid, relative to other fluids in the column, the closer to the bottom of the column the fluid will be located as defined or established by gravity. For example, the first fluid layer can have a first density and can form a first fluid layer of the multi-fluid density gradient portion. The second fluid layer can have a second density that can be greater than a density of the first fluid layer and can form a second fluid layer of the multi-fluid density gradient portion beneath the first fluid layer. An additional fluid layer(s), e.g., third, fourth, etc., can have a third, fourth, etc., densities that can be greater than a density of the previous fluid layer and can form a third, fourth, etc., fluid layer of the multi-fluid density gradient portion beneath the second fluid layer. As a note, this is not the case for the “capillary force-supported interface.” In that instance, the surface tension of the fluid relative to the size and material of the vessel provides the ability to put less dense fluids beneath fluids of greater density, e.g., below the multi-fluid density gradient portion (see FIG. 1) or above the multi-fluid density gradient portion but under another fluid of greater density (see FIG. 2).

In further detail regarding the vessels that can be used to support the vertically layered fluid column used, they can be configured as shown in FIGS. 1-5 and 7 herein or can have other shapes. In one example, the vessel can include a conical chamber (for the multi-fluid density gradient portion) which can be coupled to a round cross section capillary tube (for the capillary force gradient portion), but either or both could include a round, square, triangle, rectangle, or other polygonal cross-section with an appropriate capillary junction. Both could include bifurcations or other structures within the vessel. The vessel may include one or more expansions and constrictions and/or may include a one-to-one, one-to-many, many-to-one, or many-to-many relationship between multi-fluid density gradient portions and capillary force gradient portions. The vessel may likewise include one or more input, output, or vent ports, and may or may not be symmetrical. Furthermore, the vessel can be made of various polymers (e.g. Polypropylene, TYGON, PTFE, COC, others), glass (e.g. borosilicate), metal (e.g. stainless steel), or a combination of materials. Additionally, the capillary force gradient portion could be formed from multiple materials used in various microfluidic devices, such as silicon, glass, SU-8, PDMS, a glass slide, a molded fluidic channel(s), 3-D printed material, and/or cut/etched or otherwise formed features. Film layers can likewise be used for the structure as well. In further detail, the vessel may be monolithic or a combination of components fitted together. The vessel can be standalone or a component of a system (manual or automated) that includes functions or features for fluid positioning, particle manipulation, analysis, and/or other processes.

In further reference to the multi-fluid density gradient portion of the vertically layered fluid column, in some examples, a density of a fluid in a fluid layer can be altered using a densifier. Example densifiers can include sucrose, polysaccharides such as FICOLL™ (commercially available from Millipore Sigma (USA)), C₁₉H₂₆I₃N₃O₉ such as NYCODENZ® (commercially available from Progen Biotechnik GmbH (Germany)) or HISTODENZ™, iodixanols such as OPTIPREP™ (both commercially available from Millipore Sigma (USA)), or combinations thereof. In one example, a density difference of the first fluid layer relative to the second fluid layer can be from about 50 mg/mL to about 3 g/mL. In yet other examples, a density difference from the first fluid layer relative to the second fluid layer can be from about 50 mg/mL to about 500 mg/mL or from about 250 mg/mL to about 1 g/mL. In further detail, example additives that can be included in the first fluid layer, or in other fluid layers, depending on the design of the multi-fluid gradient column may include sucrose, C1-C4 alcohol, e.g., isopropyl alcohol, ethanol, etc., which can be included to adjust density, and/or to provide a function with respect to biological components or materials to pass through the column.

A quantity of fluid layers in the multi-fluid density gradient portion and/or the capillary force-supported gradient portion is not particularly limited. In one example, the multi-fluid density gradient portion can further include a fourth fluid layer having a fourth fluid density that can be greater than a third fluid with a third fluid density and can be positioned beneath the third fluid layer. The fourth fluid layer can be phase separated from the third fluid layer along a third fluid layer interface where the third fluid layer can be in contact as a layer relative to the fourth fluid layer. In further examples, the assembly can further include a fifth, sixth, or seventh fluid layer that can be phase separated from the other fluids in the column based on a density of the fifth, sixth, or seventh fluid with respect to the other fluids in the column.

### Biological Component Processing Systems

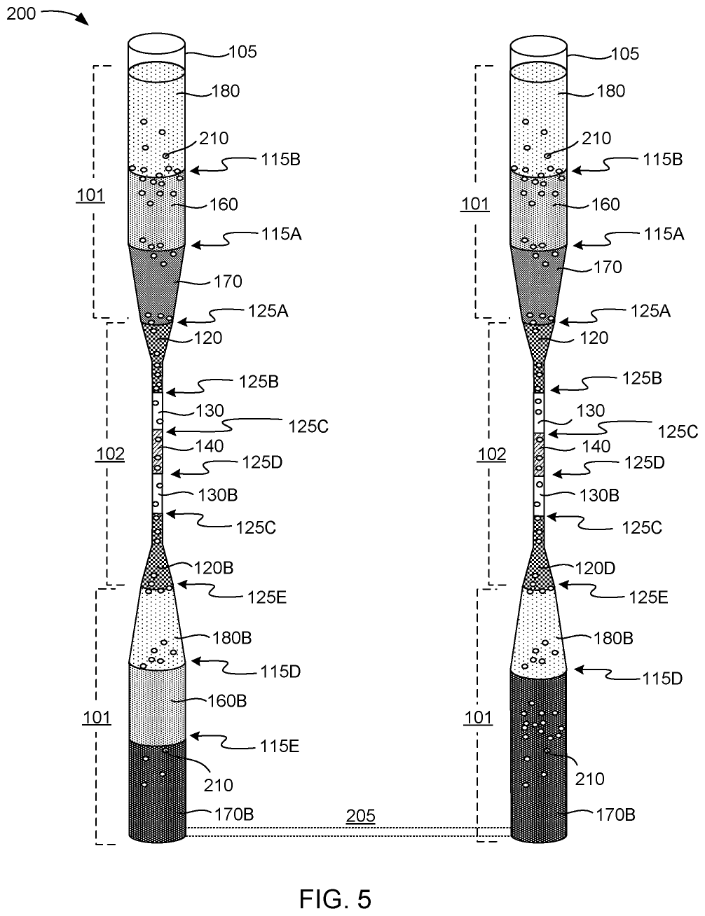

In accordance with examples of the present disclosure, a few example biological component processing systems 200 are shown in FIGS. 3-5. More specifically, in FIGS. 3-5, there is a vertically layered fluid column 100 including two portions, namely a multi-fluid density gradient portion 101 and a capillary force gradient portion 102 (in the case of FIG. 5, there are two multiple columns assembled together in series). The two portions (or more in the case of FIG. 5) may be contained or supported by a vessel 105 as previously described.

Referring initially to FIG. 3 more specifically, the system can include a vertically layered fluid column 100, similar to that shown by example in FIGS. 1 and 2. This particular vertically layered fluid column is more complicated by example, but is used to shown the versatility of the fluid columns and systems, and not by way of limitation. The multi-fluid density gradient portion includes two (or more) individual fluids of different densities. In this example, fluid 160 has a first fluid density, fluid 170 has a second fluid density that is greater than the first fluid density, and fluid 180 (top fluid) has a fluid density that is less than the first fluid density.

The density difference between fluid 160 and fluid 170 can be sufficient so that the fluids remain phase separated at a first density-differential interface 115A, and the density difference between the fluid 170 and fluid 180 can be sufficient so that the fluids remain phase separated at a second density differential interface 1156, even though the fluids are in direct contact or in direct fluid communication layered on top of one another, e.g., the fluids are separated by their densities, not by a membrane or other artificial structure therebetween. Example density differences of any two fluids along the multi-fluid density gradient portion can be from 50 mg/mL to 3 g/mL, from 100 mg/mL to 3 g/mL, from 500 mg/mL to 3 g/mL or from 1 g/mL to 3 g/mL.

The vertically layered fluid column 100 also includes fluid 120 in this example. However, in this instance, the density of fluid 120 is less than the fluid 170, even though it is positioned immediately there beneath. Thus, in a more standard sized column, fluid 120 may otherwise migrate up into or through the second fluid 170, destroying the interface between the fluids. However, in the example shown, this is not the case, as fluid 120 is constrained by the cross-sectional size of the column structure (along the plane where the second fluid interfaces with the third fluid). Thus, the surface tension of fluid 120 combined with the size constraint of the column at this interface provides a (first) capillary force-supported interface 125A, which promotes fluid 170 and fluid 120 remaining separated from one another. More specifically, the capillary force-supported interface can be contained within a fluidic channel having a cross-sectional dimension that is aligned with the capillary force-supported interface, and can be, for example, less than 2 mm, from 1 μm to 1 mm, from 1 mm to 1.75 mm, from 0.75 μm to 100 μm, or from 1 μm to 50 μm. This dimension can be a diameter dimension, or for non-circular geometries, this dimension can be the average cross-sectional dimension. Again, fluid 160, fluid 170, fluid 120, or any of the other fluids can be any of a number of combinations of fluids, such as gas fluid, e.g., air, aqueous fluid, non-polar fluid, polar, non-polar, miscible, immiscible, etc. The fluids can be, for example a master mix fluid, reagent fluid, surfacing binding fluid, washing fluid, elution fluid, lysis fluid, etc. The fluids can likewise be pure, solutions, mixtures, suspensions, emulsions, and/or in other forms. They may or may not undergo chemical reactions within the vessel at any stage of the process, depending on the application.

In further detail, in the example shown in FIGS. 3 and 4, fluid 130 can be even less dense than fluid 120, and thus, at the interface therebetween, there can be a second capillary force-supported interface 125B. In fact, fluid 130 may be a gas, such as air, for example, which is less dense than the fluid 120, which may be an oil or some other lubricating fluid that may promote the movement of the particulate substrates across the gaseous gap, such as an air gap by way of example, provided by fluid 130. Fluid 140, on the other hand, may be denser than fluid 130. In this instance, the interface can be in the form of a third capillary force-supported interface 125C and because of the greater density, can also be described as a density-differential interface 115C. In this example, fluid 140 can be any fluid that could act on a biological component found on a particulate substrate, e.g., magnetizing particles, that has been pre-processed, e.g., lysed cells, washed component, etc. For example, fluid 140 can be a master mix for biological processing or an elution buffer for elution of the biological component.

In further detail, individual fluids in the various layers can provide different functions, regardless of the orientation, e.g., FIG. 3 or FIG. 4 orientation. For example, a fluid in one layer can include a lysis buffer to lyse cells. In yet other examples, a fluid of another layer can be a surface binding fluid to bind the biological component to the magnetizing particles, a wash fluid to trap contaminants from a sample fluid and/or remove contaminants from an exterior surface of the magnetizing particles, a surfactant fluid to coat the magnetizing particles, a dye fluid, an elution fluid to remove the biological component from the magnetizing particles following extraction from the biological sample, a labeling fluid for binding labels to the biological component such as a fluorescent label (either attached to the magnetizing particles or unbound thereto), a reagent fluid to prep a biological component for further analysis such as a master mix fluid to prep a biological component for PCR, and so on.

In some examples, an individual fluid in one or multiple layers can provide sequential processing of a biological component from a biological sample. For example, individual fluids can carry out individual functions, and in many cases, the functions can be coordinated to achieve a specific result. Biological samples that may be added can include whole blood, platelets, cells, lysed cells, cellular components, tissue, nucleic acids, e.g., DNA, RNA, primers, oligos, etc., or poly-bases, peptides, or the like. More specifically, for example, in considering biological components of interest and second components found in a cell, sequential fluid from top to bottom of a multi-fluid density gradient portion can act on the cell to lyse the cell in one of the fluids, and bind a target biological component from the lysed cell to particulate substrate, e.g., magnetizing particles, in a second fluid (or lysing and binding can alternatively be done in a single fluid). Additional fluid may be used to wash the particulate substrate with the biological component bound thereto in another fluid, e.g., washing the second fluid from particulate substrate in the next fluid, and/or eluting (or separating) the biological component from the particulate substrate in yet another lower layer. The surface binding and cell lysis can occur, for example, with a lysate buffer in a sucrose and water solution, e.g., the lysate (lysis) buffer can be densified with sucrose. Washing can occur in a sucrose in water solution, for example. In other examples, one or more of the fluids can be present as a fluid (layer(s)) along the multi-fluid density gradient portion in the form of a master mix fluid for nucleic acid processing. Other combinations of fluids (first, second, third, etc.) may include a surfacing binding fluid, a washing fluid, and an elution fluid; or may include a lysis fluid, a washing fluid, a surface binding fluid, a second washing fluid, an elution fluid, and a reagent fluid. Regardless of the various functions of the various fluids with sequentially increasing densities arranged from top to bottom, at the individual fluids, the particulate substrate can independently interact, e.g., become modified, with fluids as layers in order to sequentially process the particulate substrate with surface active groups and/or biological components associated therewith or associated with one or more of the fluids, for example.

A vertical height of the various layers of fluids in the multi-fluid density gradient portion can vary. Adjusting a vertical height of a fluid layer can affect a residence time of the magnetizing particles, e.g., paramagnetic microparticles, in that fluid layer. The taller the fluid layer, the longer the residence time of the magnetizing particles in the fluid layer. Notably, the speed at which the magnets move may also be adjustable. In some examples, all of the fluid layers in the multi-fluid density gradient portion can be the same vertical height. In other examples, a vertical height of individual fluid layers in a multi-fluid density gradient portion can vary from one fluid layer to the next. In one example, a vertical height of the various layers along the multi-fluid density gradient portion can individually be from about 10 μm to about 50 mm. In another example, a vertical height of the fluid layers along the multi-fluid density gradient portion can individually be from about 10 μm to about 30 mm, from about 25 μm to about 1 mm, from about 200 μm to about 800 μm, or from about 1 mm to about 50 mm.

The biological component processing systems 200 shown in FIGS. 3-5, can further include a particulate substrate 210 to pass through the density-differential interface and the capillary force-supported interface. Referring specifically to FIG. 3 and FIG. 4, these systems can include particulate substrate (or particles) of a sufficient density (greater density than the density of some or all of the fluid layers) to pass through the vertically layered fluid column, or interfaces thereof, by gravity, centrifugation, etc. For example, the density of the particulate substrate can be sufficient to allow the particulate substrate to pass through the density-differential interface and the capillary force-supported interface either by the force of gravity or centrifugation. Alternatively, though not shown specifically in FIG. 3, but shown in FIG. 4 with a few example magnets 190, the particulate substrate can include magnetizing particles, and the fluid column further includes magnet(s) to move the magnetizing particles along a z-axis through the density-differential interface and through the capillary force-supported interface. The magnet(s) can be in the form of permanent magnet(s), or can be electrically induced magnetic elements, for example. The magnets can be moved vertically to move magnetizing particles vertically (along the z-axis), though there can also be movement along the x- and y-axes as well. In still other examples, there may be a vertical array of electrically induced magnets that can be sequentially or serially induced or activated to move the magnetizing particles along the column, for example.

In further detail regarding the “particulate substrate,” these materials can be in the form of particles, such as microparticles. These particles can be denser than the various fluids along the vertically layered fluid column so that they move in a negative z-axis direction, e.g., glass, silica, etc., or can be less dense or buoyant so they can move upward through the vertically layered fluid column in a positive z-axis direction. The particulate substrate can also be particles that are “magnetizing” meaning that when a magnetic field is applied, they can respond and move with the magnetic field when a static magnetic field is applied and/or can move as the magnetic field is modified dynamically. Movement of the magnetizing particles can be a positive or negative z-axis direction. The surface morphology of the particulate substrate may be smooth or rough, and dimensions may vary from sub-micron, e.g., from 100 nm or 500 nm, to about 20 μm. The particulate substrate may, for example, include surfaces that bind to nucleic acids either reversibly or permanently (relative to downstream processing fluids selected for use). The particulate substrate may be in the form of glass or silica particles May bind NA reversibly or permanently, based on downstream process requirements.

The particulate substrate can include surfaces that are bound to or otherwise associated with a biological component or can be formulated to become bound to or otherwise associated with a biological component in situ.

The term “associated” refers to any type of attach or adherence of a biological component with a surface of the particulate substrate. This can include covalent bonding, electrostatic or ionic attraction, surface adsorption, hydrogen bonding, and/or other adherence or linkage suitable for moving biological component together with the particulate substrate. In accordance with this, in some examples, the system can include a biological sample and the particulate substrate (magnetizing particles or otherwise) can be surface activated to preferentially bind with a biological component relative to secondary components in a biological fluid sample. Thus, the biological component may preferentially bind to the surface compared to secondary components such as enzymes, cellular debris, lysing agents, buffers, or a combination thereof. The particulate substrate can be loaded in any of the fluid layers and moved vertically in either direction (up or down). As shown in FIG. 3, the fourth fluid 180 is shown as being pre-loaded with the particulate substrate. As shown in FIG. 4, the particulate substrate is shown as being loadable into the fourth fluid. Pre-loading or loadability can be in any of the various layers in addition to that specifically shown. Also shown in FIG. 4, there are some magnetizing particles shown that are depicted as being gathered near a magnet, which can be used to move the magnetizing particles upward or downward along the z-axis of the vertically layered fluid column, for example. There are many other magnet orientations, spatial locations, etc., that can be used to move magnetizing particles across the various types of fluid interfaces.

In further detail regarding the particulate substrate, as mentioned, the particulate substrate can be particles with a density suitable for gravity settling or centrifugation separation or movement along the column. In some examples, the particulate substrate can be in the form of magnetizing particles. Magnetizing particles can be in the form of paramagnetic microparticles, superparamagnetic microparticles, diamagnetic microparticles, or a combination thereof, for example. Whether using magnetizing particles or otherwise, the particulate substrate can be surface-activated to bind with a biological component or can be bound to the biological component. The particulate substrate can be surface activated, for example, with surface groups that are interactive with a biological component of a biological sample or can include a covalently attached ligand attached to a surface of the particulate substrate to likewise bind with a biological component of a biological sample. In some examples, the ligand can include proteins, antibodies, antigens, nucleic acid primers, amine groups, carboxyl groups, epoxy groups, tosyl groups, sulphydryl groups, silane groups, poly dT oligomers, target-specific oligomers, streptavidin, or the like.

Regarding combinations of ligands, there can be multiple types of ligands on a common particulate substrate, a mixture of particulate substrate with different ligands on multiple portions of the particulate substrate (with the same or different particulate substrate). The ligand can be selected to correspond with and bind with the biological component and can vary based on the type of biological component being isolated from the biological sample. For example, the ligand can include a nucleic acid primer when isolating a biological component that includes a nucleic acid sequence. In another example, the ligand can include an antibody when isolating a biological component that includes antigen. By way of example, commercially available examples of magnetizing particles with surface-activated groups include those sold under the trade name DYNABEADS®, available from ThermoFischer Scientific (USA).

In some examples, the particulate substrate can have an average particle size that can be from about 0.1 μm to about 70 μm. The term “average particle size” morphology of the individual particle. A shape of the particulate substrate can be spherical, irregular spherical, rounded, semi-rounded, discoidal, angular, sub-angular, cubic, cylindrical, or any combination thereof. In one example, the particles can include spherical particles, irregular spherical particles, or rounded particles. The shape of the particulate substrate can be spherical and uniform, which can be defined herein as spherical or near-spherical, e.g., having a sphericity of >0.84. Thus, any individual particles having a sphericity of <0.84 are considered non-spherical (irregularly shaped). The particle size of the substantially spherical particle may be provided by its diameter, and the particle size of a non-spherical particle may be provided by its average diameter (e.g., the average of multiple dimensions across the particle) or by an effective diameter, e.g., the diameter of a sphere with the same mass and density as the non-spherical particle. In further examples, the average particle size of the particulate substrate can be from about 1 μm to about 50 μm, from about 5 μm to about 25 μm, from about 0.1 μm to about 30 μm, from about 40 μm to about 60 μm, or from about 25 μm to about 50 μm.

In an example, the particulate substrate can be unbound to a biological component when added directly to one of the fluid (layers) of a multi-fluid density gradient portion and/or the capillary force gradient portion. Binding between the particulate substrate and the biological component of the biological sample can occur in the multi-fluid density gradient portion and/or the capillary force gradient portion. In yet another example, the particulate substrate and a biological sample including a biological component can be combined in a loading fluid before being added to a multi-fluid density gradient portion. In this example, binding of the particulate substrate to the biological component of the biological sample can occur in the multi-fluid density gradient portion.

With more specific detail regarding the magnetizing particles, the term “magnetizing particles” is defined herein to include microparticles that may not be magnetic in nature unless and until a magnetic field is introduced at a strength and proximity to cause them to become magnetic. Their magnetic strength can be dependent on the magnetic field applied and may get stronger as the magnetic flied is increased, or the magnetizing particles get closer to the magnetic source that is applying the magnetic field. In more specific detail, “paramagnetic microparticles” have these properties, in that they have the ability to increase in magnetism when a magnetic field is present; however, paramagnetic microparticles are not particularly magnetic when a magnetic field is not present. In some examples, the paramagnetic microparticles can exhibit no residual magnetism once the magnetic field is removed. A strength of magnetism of the paramagnetic microparticles can depend on the strength of the magnetic field, the distance between a source of the magnetic field and the paramagnetic microparticles, and a size of the paramagnetic microparticles. As a strength of the magnetic field increases and/or a size of the paramagnetic microparticles increases, the strength of the magnetism of the paramagnetic microparticles increases. As a distance between a source of the magnetic field and the paramagnetic microparticles increases the strength of the magnetism of the paramagnetic microparticles decreases. “Superparamagnetic microparticles” can act similar to paramagnetic microparticles; however, they can exhibit magnetic susceptibility to a greater extent than paramagnetic microparticles in that the time it takes to become magnetized appears to be near zero seconds. “Diamagnetic microparticles,” on the other hand, can display magnetism due to a change in the orbital motion of electrons in the presence of a magnetic field.

The vertically layered fluid column can be part of a system that includes magnetizing particles, which can be, for example, paramagnetic microparticles, superparamagnetic microparticles, diamagnetic microparticles, or a combination thereof. Paramagnetic microparticles can have the ability to increase in magnetism when a magnetic field is present; however, paramagnetic microparticles are not magnetic when a magnetic field is not present. In some examples, the paramagnetic microparticles can exhibit no residual magnetism once the magnetic field is removed. The strength of magnetism of the paramagnetic microparticles can depend on the strength of the magnetic field, the distance between a source of the magnetic field and the paramagnetic microparticles, and the size of the paramagnetic microparticles. As the strength of the magnetic field increases and/or the size of the paramagnetic microparticles increases, the strength of the magnetism of the paramagnetic microparticles will be larger. As the distance between the source of the magnetic field and the paramagnetic microparticles increases, the strength of the magnetism of the paramagnetic microparticles decreases.

Superparamagnetic microparticles can act similar to paramagnetic microparticles; however, they can exhibit magnetic susceptibility more quickly than paramagnetic microparticles in that the magnetization time appears to be near zero seconds. Diamagnetic microparticles can display magnetism due to a change in the orbital motion of electrons in the presence of a magnetic field.

Referring now to FIG. 5, an alternative biological component processing system 200 is shown that includes one or multiple vertically layered fluid columns 100. The columns shown include an inverted portion of a column (similar to that shown in FIG. 2) connected in series with another portion that is similar to that shown in FIGS. 1, 2 and 4. Furthermore, also shown in FIG. 5, a fluidic channel 205 is shown that may, in some examples, be used to interconnect multiple vertically layered fluid columns. In other examples, the horizontal fluidic channel shown can also be a capillary tube which keeps fluids separate using capillary forces as well. The two vertically layered fluid columns shown in FIG. 5 can alternatively be considered to be independent of one another, which is why the fluidic channel is shown in phantom lines.

In further detail regarding FIG. 5, both columns shown include two multi-fluid density gradient portions 101 separated by a capillary force gradient portion 102. The two columns shown are slightly different, but it is noted that they can be more significantly different than shown by selecting different fluids, different fluid densities, different fluid layer heights, different column structure, etc. With this in mind, both vertically layered fluid columns include fluids 160, 170, and 180 in one of its multi-fluid density gradient portions, and fluids 170B and 180B in its other multi-fluid density gradient portion. One of the columns further includes fluid 160B as well, which is an additional fluid compared to the other column. Both columns also include several density-differential interfaces, shown at 115A-115E. Additionally, both columns also include multiple capillary force-supported interfaces, shown at 125A-125E. Again, any of the fluids in these layers can be any of the fluid layers described herein, but at the multi-fluid density gradient portions of the columns, the various fluids can be separated due to their density difference, and in the capillary force gradient portion, the various fluids include one or more capillary force-supported interface, though there may also be density-differential interfaces within the capillary force gradient portion, as shown at 125C. At this interface, there is density-differential interface that is present due to the density difference of the two fluids, e.g., oil at 140 positioned beneath air 130), and this density difference is sufficient to provide the interface even if the fluids were not present in the capillary force gradient portion. Thus, in order for a capillary force-supported interface to be present, the two fluids along the interface would not otherwise remain as separate layers were it not for the constrained nature of the vessel surrounding the capillary force gradient portion, e.g., a capillary tube less than 2 mm.

Thus, in one example, as shown, the biological component processing system 200 of FIG. 5 can be summarized, without limitation, as including four multi-density gradient portions 101 separated by three capillary force gradient portions 102 (when fluidic channel 205) is included. When fluidic channel 205 is not included, the biological component processing system shown in FIG. 5 can be summarized as two independent systems, both including two multi-density gradient portions separated by a capillary force gradient portion. As an example of how particulate substrates might be processed using this type of system, the particles could first be moved in a downward (or negative) z-axis direction, then horizontally through fluidic channel 205, and then, moved in an upward (or positive) z-axis direction through the other column. This could occur in either direction starting with one or the other column. Many permutations of this concept are possible including those that move the particulate substrate back and forth between treatments, and those that include one or more ports along the fluidic pathway of the particles for adding or removing fluids and/or particulate substrates, e.g., harvesting and/or additive treatments.

The vertically layered fluid columns and/or biological component processing systems described herein can be used or can further include a magnet, such as in the form of electrically induced magnet field generating element(s), permanent magnet(s), or a combination thereof. An electrically induced magnetic element can be, for example, turned on and off by introducing electrical current/voltage to the element and generating a magnetic field. Alternatively, the magnet can be a permanent magnet that is placed in proximity to the multi-fluid density gradient portion to effect and/or affect the movement of the magnetizing particles. In either instance, the magnet can be moved physically to cause movement of magnetizing particles along the vertically layered fluid column. Thus, magnet can be permanently placed within this proximity, or can be movable along the column, or movable in position and/or out of position to effect movement of the magnetizing particles. The magnetizing particles can be magnetized by the magnetic field generated by the magnet or electrically induced magnet. In addition, the magnet can create a force capable of pulling the magnetizing particles through the multi-fluid density gradient portion. When the magnet is turned off or is not in appropriate proximity, the magnetizing particles can reside in a fluid layer until gravity pulls the magnetizing particles through fluid layers of the multi-fluid density gradient portion, or they may remain suspended in the fluid layer in which they may reside until the magnetic field is applied thereto. The rate at which gravity pulls the magnetizing particles through fluid layers (or leaves the magnetizing particles within a fluid layer) can be based on a mass of the magnetizing particles in combination with a surface tension between fluid layers and/or density of the magnetizing particles and density of the fluid layers. The magnet can cause the magnetizing particles to move from one fluid layer to another or increase a rate at which the magnetizing particles pass from one fluid layer into another.

In an example, the magnet 190 can be positioned above (FIG. 2), below (FIG. 4), or along the side (FIG. 4) of the multi-fluid density gradient portion. The magnet can be in a fixed position or can be moveable in position, out of position, or at variable positions to effect downward movement, rate of movement, or to promote little to no movement of the magnetizing particles. In another example, the magnet can be positioned adjacent to a side of the multi-fluid density gradient portion and can move vertically to cause the magnetizing particles to move therewith. In some examples, the magnet can be a ring magnet, as shown in FIG. 4. A movable magnet(s) can likewise be positioned adjacent to a side of the multi-fluid density gradient portion that is not a ring shape but can be any shape effective for moving magnetizing particles along the column. In some examples, the magnet can be moved along a side and/or along a bottom of the multi-fluid density gradient portion to pull the magnetizing particles in one direction or another. In one example, the magnet can be used to pull the magnetizing particles downwardly through fluid layers of the multi-fluid density gradient portion. In yet other examples, the magnet can be used to concentrate the magnetizing particles near a side wall of the multi-fluid density gradient portion to be moved downward by a movable magnet, or by a magnet positioned beneath the multi-fluid density gradient portion. In one example, a magnet used to move magnetizing particles downward can be used to reverse the direction of the magnetizing particles and can cause the magnetizing particles to re-enter a fluid layer that the magnetizing particles have previously passed through.

A strength of the magnetic field and the location of the magnet in relation to the magnetizing particles can affect a rate at which the magnetizing particles move downwardly through the multi-fluid density gradient portion. The further away the magnet and the lower the strength of the magnetic field, the slower the magnetizing particles will pass through the multi-fluid density gradient portion. In an example, a maximum distance between the magnet and a nearest location where one or more of the fluids resides along the multi-fluid density gradient portion can be about 50 mm maximum distance, about 40 mm maximum distance, about 30 mm maximum distance, about 20 mm maximum distance, or about 10 mm maximum distance. The minimum distance, on the other hand, may be from about 0.1 mm minimum distance, from about 1 mm minimum distance, or from about 5 mm minimum distance. In one example, the minimum distance between the magnet and the multi-fluid density gradient portion may be about the thickness of the vessel that contains the multi-fluid density gradient portion. Thus, distance ranges between the magnet and the multi-fluid density gradient portion can be from about 0.1 mm to about 50 mm, from about 1 mm to about 50 mm, from about 1 about mm to about 40 mm, from about 1 mm to about 30 mm, from about 1 mm to about 20 mm, from about 1 mm to about 10 mm, from about 5 mm to about 50 mm, or from about 5 mm to about 30 mm. In another example, a maximum distance between the magnet and a nearest location where one of the fluids resides along the multi-fluid density gradient portion can be about 30 mm.

Methods of Processing Biological Components from Biological Samples

A flow diagram of a method 300 of processing a biological component from a biological sample is shown in FIG. 6, which can include loading 310 a biological sample into a vertically layered fluid column including a plurality of fluids positioned in fluid layers, a density-differential interface along which two fluids from the plurality of fluids are positioned, and a capillary force-supported interface along which two fluids from the plurality of the fluids are positioned. The method in this example also includes passing 320 a biological component of the biological sample through the density-differential interface and the capillary force-supported interface

In one example, the biological component can be associated with a particulate substrate that is passed through the density-differential interface and the capillary force-supported interface via a force selected from gravity, a centrifugal force, a magnetic field, buoyance, or a combination thereof. In another example, the biological component can be associated with a particulate substrate that is passed through the density-differential interface and the capillary force-supported interface via a magnetic field. The term “processing” can refer to concentrating, separating, diluting, amplifying, washing, lysing, decontaminating, fluid exclusion, eluting, or carrying out any other process where the biological component is modified in some way within the vertically layered fluid column.

In one example, the biological component can be bound to or otherwise associated with a particulate substrate that is passed through the density-differential interface and the capillary force-supported interface via a force selected from gravity, a centrifugal force, a magnetic field, buoyancy, or a combination thereof. In some other examples, the biological sample including the biological component can be combined with the magnetizing particles in a loading solution prior to loading the biological sample including the biological component and the magnetizing particles into the multi-fluid density gradient portion. For example, the magnetizing particles and the biological sample can be admixed in a loading fluid. The biological sample and the magnetizing particles can be permitted to incubate or otherwise become prepared for loading on top of or into the multi-fluid density gradient portion. The magnetizing particles can bind with the biological component in the loading fluid and can then be added to the multi-fluid density gradient portion for the fluid layers to act upon the magnetizing particles. In one example, the loading fluid can become the uppermost fluid layer when loading from the top or can become the lowermost fluid layer when loading from the bottom, for example. Other fluid layers beneath or above the loading layer can be included through which the particulate substrate is passed in part or in full.

The fluid used for loading the column (or the first fluid, or even the second fluid, or other fluid layer) can include secondary components selected from enzymes, cellular debris, lysing agents, buffers, or a combination thereof. The magnetizing particles can be bound to the biological component in a loading fluid or in a subsequent fluid along the multi-fluid density gradient portion. In the case of a loading fluid, magnetizing particles including the biological component bound thereto can then be introduced as a separate fluid layer for the microparticles to be drawn into other fluid layers that can act on the microfluidic particles to further interact with the surface thereof along the multi-fluid density gradient portion.

In accordance with the method, the particulate substrate can be passed through multiple density-differential interfaces and/or multiple capillary force-supported interfaces, depending on the arrangement. The particulate substrate can be passed through any or all of these interfaces from fluid to fluid in an upward or downward z-axis direction, though movement along the x- and y-axes can also occur during the movement of the particles upward or downward. It is noted once again that the vertically layered fluid column can be arranged with layered fluids in an orientation of about 90 degrees from horizontal, or the column can be at any angle suitable for upward or downward movement of the particulate substrate through horizontal fluid interfaces.

In one example, the method can further include selectively withdrawing, e.g., pipetting, the biological component out of the third fluid layer, such as through an ingress/egress opening(s) from the top, the bottom, or through a sidewall, for example. The biological component may still be associated with a surface of the magnetizing particles or may be separated from the magnetizing particles. In another example, this method alternatively may include selectively withdrawing, e.g., pipetting, from one of the fluids (one of the layers), the second fluid layer, and/or the third fluid layer out of the multi-fluid density gradient portion and leaving the magnetizing particles with the biological component bound thereto in a vessel of the multi-fluid density gradient portion to either be further treated or removed after the extraction of one of the fluids, the second fluid layer, and the third fluid layer therefrom. In some examples, the biological sample can include a cell with the biological component trapped within the cell (prior to lysis), a virus, or a biological component with extra-cellular vesicles. Lysing the cell can release the biological component therefrom and can permit isolation of the biological component. In this example, one of the fluids (or a loading fluid) can include a lysing agent for the cell. The method can further include lysing the cell in situ within one of the fluids or the loading fluid so that the biological component can be liberated from the cell and can bind with the magnetizing particles in one of the fluids (or fluid layers) or the loading fluid.

FIG. 7 illustrates one specific example of how the vertically layered fluid columns and biological component processing system can be used in accordance with the methods described herein. This example is provided by way of example and is not to be considered as limiting. In FIG. 7, a system is shown similar to that shown in FIGS. 3 and 4. This method can be carried out using magnetizing particles, or particles having a density sufficient to flow through the vertically layered fluid column. Specifically, the vertically layered fluid column can include two portions, a multi-fluid density gradient portion and a capillary force gradient portion, as previously described. In this example, fluid 160 has a first fluid density, fluid 170 has a second fluid density that is greater than the first fluid density, and fluid 180 (top fluid) has a fluid density that is less than the first fluid density. These fluids make up the multi-fluid gradient portion of the column. The density difference between fluid 160 and fluid 170 can be sufficient so that the fluids remain phase separated at a first density-differential interface 115A, and the density difference between the fluid 170 and fluid 180 can be sufficient so that the fluids remain phase separated at a second density differential interface 115B. The column also includes fluid 120, which has a density that is less than fluid 170, even though it is positioned immediately there beneath. As described, the surface tension of fluid 120 combined with the size constraint of the column at this interface provides a (first) capillary force-supported interface 125A, which promotes fluid 170 and fluid 120 remaining separated from one another. Fluid 130 can be a gas, for example, with a second capillary force-supported interface there above. Fluid 140 can be a liquid, and thus the interface there above can be provided by a fluid density differential 125C, even though it is present along the capillary force gradient portion, as previously described.

As mentioned, fluid 160, fluid 170, fluid 120, or any of the other fluids can be any of a number of combinations of fluids, such as gas fluid, e.g., air, aqueous fluid, non-polar fluid, master mix fluid, reagent fluid, surfacing binding fluid, washing fluid, elution fluid, lysis fluid, etc. However, in this example, various processes are shown in sequence to provide an example of the use of the present technology. Notably, magnets are not shown, but if magnetizing particles are used as the particulate substrate, then a magnet or series of magnets can be used to draw the particles in a controlled manner in the negative z-direction.

In accordance with the general processing stream shown in FIG. 7, a more specific example may include lysing and particle binding (A) as cells, viral particles, or the like (shown at 250) and are initially lysed and a nucleic acid 220 is released in fluid 180. The lysis-binding in fluid 180, for example, can be carried out using a solution including one or more of:
guanidine salt-based or other high salt content buffers used for solid phase extraction;
alcohol such as isopropyl alcohol (IPA), ethanol (EtOH), polyethylene glycol (PEG), or other suitable alcohols;
carrier nucleic acid(s);
enzymes to assist in lysis, such as Proteinase K, for example; and/or
pH adjuster to modify pH.

The nucleic acid from the cell can become bound to a surface of the particulate substrate 210, which can be a magnetizing particle (M), for example. Next, shown at (B), as the particles are drawn through interface 115B, cellular debris and unbound nucleic acid can be exchanged for a wash buffer at fluid 160. A second wash can occur at fluid 170, shown at (C), as bead-bound nucleic acid is further cleared of contaminants, e.g., lysed cellular debris and/or other contaminants or other material not of interest that may be present. Example wash buffers for use at fluids 180 and/or 160 can be:
an aqueous solution including an alcohol, such as ethanol, and one or more of another alcohol, a binding agent binding agent, a salt, a surfactant, and/or a stabilizing agent; and/or
MyOne™ silane genomic DNA or viral kits, mRNA Direct kits, Mag Max™ kits, or other similar kits that include wash buffers often used with DYNABEADS®, all available from ThermoFischer Scientific, USA.

In further detail regarding the specific example shown in FIG. 7, along the capillary force gradient portion of the vertically layered fluid column, in this example, there are two fluids that can work together to clear debris from a particulate substrate as it is moved from the multi-fluid density gradient column portion (after the two layers of wash buffer) and into the capillary force gradient portion of the column. Those two fluids include the use of an oil and a gas. The oil can be, for example, mineral oil. The gas can be, for example, air. Thus, the oil can be used for oil exclusion, which is shown as being carried out at (D), where aqueous solution that may be present on or even entrapped in the particulate substrate (or magnetizing particles) can be replaced with the oil. Other fluids may be suitable for this, but mineral oil is a good example of a fluid that may benefit from phase separation due to capillary forces in accordance with the present disclosure. Furthermore, by using an oil, this can provide an effective way of transitioning the particulate substrate from being carried by a liquid fluid and passed into a gaseous fluid, such as air, as shown at (E).

Regarding the oil layer in the capillary force gradient portion of the column, shown at (D), specific oils that can be used include:
light oils, such as mineral oil for molecular biology or molecular grade mineral oils, light oil M5904 (density 0.84 g/mL at 25° C.) from Sigma-Aldrich (USA);
olive oil, such as high purity olive oil; and/or
densified oil.

Once the particulate substrate passes through the oil, a gas layer, which in this instance can be air (or an air gap), can be used to clear the contaminants further and can provide a mechanism to provide little to no contact between the fluids above and below the air gap. The biological component being separated (or further processed) has now have been loaded on the particulate substrate after cell lysis, washed in two different wash buffer layers, further contaminant-cleared by the oil, and passed through the air gap, providing reduced likelihood of concentration of contact between wash and/or lysis buffer and the next fluid beneath the air gap, e.g., elution buffer, a master mix fluid for nucleic acid processing, or the like. To separate the biological component, e.g., nucleic acid, from the particulate substrate, an elution buffer can be used. Example elution buffers suitable for use may include one or more of:
aqueous salt solution (sufficient for elution but to retain biological component intact);
stabilizers;
surfactants; and/or
master mix if it is for a direct elution process, provide column is tuned for a target biological component of interest.

### Definitions

It is noted that, as used in this specification and the appended claims, the singular forms “a,” “an,” and “the” include plural referents unless the context clearly dictates otherwise.

As used herein, the term “about” is used to provide flexibility to a numerical range endpoint by providing that a given value may be “a little above” or “a little below” the endpoint. The degree of flexibility of this term can be dictated by the particular variable and determined based on experience and the associated description herein.

As used herein, the term “interact” or “interaction” as it relates to a surface of the particulate substrates, such as the magnetizing particles, and indicates that a chemical, physical, or electrical interaction occurs where a particulate substrate surface property is modified in some manner that is different than may have been present prior to entering the fluid layer, but does not include modification of magnetic properties magnetizing particles as they are influenced by the magnetic field introduced by the magnet. For example, a fluid layer can include a lysis buffer to lyse cells, and cellular components can become bound to or otherwise associated with a surface of the magnetizing particles. Lysing cells in a fluid can modify the fluid sample and thus modify or interact with a surface of magnetizing particles, e.g., the cellular component binds or becomes otherwise associated with a surface of the magnetizing particles. In one example, the association between the biological component and the magnetizing particles (or other particulate substrate) can alternative include surface adsorption, electrostatic attraction, or by some other attraction between biological component and the surface of the particulate substrate. In yet other examples, a fluid layer that would be considered to interact with the magnetizing particles could be a wash fluid layer to trap contaminates from a sample fluid and/or remove contaminates from an exterior surface of the magnetizing particles, a surfactant fluid layer to coat the magnetizing particles, a dye fluid layer to introduce visible or other markers to the fluid or surface, an elution fluid layer to remove the biological component from the magnetizing particles following extraction from the biological sample, a labeling fluid layer for binding labels to the biological component such as a fluorescent label (either attached to the magnetizing particles or unbound thereto), a reagent fluid layer to prep a biological component for further analysis such as a master mix fluid layer to prep a biological component for PCR, and so on.

As used herein, a plurality of items, structural elements, compositional elements, and/or materials may be presented in a common list for convenience. However, these lists should be construed as though individual members of the list is individually identified as a separate and unique member. Thus, no individual member of such list should be construed as a de facto equivalent of any other member of the same list solely based on presentation in a common group without indications to the contrary.

Concentrations, amounts, and other numerical data may be expressed or presented herein in a range format. A range format is used merely for convenience and brevity and thus should be interpreted flexibly to include not only the numerical values explicitly recited as the limits of the range, but also to include individual numerical values or sub-ranges encompassed within that range as if numerical values and sub-ranges are explicitly recited. As an illustration, a numerical range of “about 1 wt % to about 5 wt %” should be interpreted to include not only the explicitly recited values of about 1 wt % to about 5 wt %, but also to include individual values and sub-ranges within the indicated range. Thus, included in this numerical range are individual values such as 2, 3.5, and 4 and sub-ranges such as from 1-3, from 2-4, and from 3-5, etc. This same principle applies to ranges reciting one numerical value. Furthermore, such an interpretation should apply regardless of the breadth of the range or the characteristics being described.

While the present technology has been described with reference to certain examples, various modifications, changes, omissions, and substitutions can be made without departing from the spirit of the disclosure. The disclosure is limited only by the scope of the following claims.

What is claimed is:

1. A vertically layered fluid column, comprising:
a plurality of fluids positioned in fluid layers;
a density-differential interface along which two fluids from the plurality of fluids are positioned; and
a capillary force-supported interface along which two fluids from the plurality of the fluids are positioned.

2. The vertically layered fluid column of claim 1, wherein one of the fluids is positioned along the density-differential interface is also positioned along the capillary force-supported interface.

3. The vertically layered fluid column of claim 1, wherein one of the fluids is an oil and is positioned along a capillary force-supported interface.

4. The vertically layered fluid column of claim 1, including four fluids positioned as fluid layers, wherein two of the fluids are positioned along the density-differential interface and the two of the fluids are positioned along the capillary force-supported interface.

5. The vertically layered fluid column of claim 4, wherein one of the fluids is a gas and one of the fluids is an oil, and the gas and the oil are positioned along the capillary force-supported interface.

6. The vertically layered fluid column of claim 1, further comprising a biological sample including a biological component to pass through the density-differential interface and the capillary force-supported interface.

7. The vertically layered fluid column of claim 1, wherein the capillary force-supported interface separates a lower fluid from an upper fluid, wherein the lower fluid is less dense than the upper fluid.

8. The vertically layered fluid column of claim 1, wherein the capillary force-supported interface is contained within a fluidic channel having a cross-sectional dimension that is aligned with the capillary force-supported interface is less than 2 mm.

9. A biological component processing system, comprising:
a vertically layered fluid column including:
a plurality of fluids positioned in fluid layers,
a density-differential interface along which two fluids from the plurality of fluids are positioned, and
a capillary force-supported interface along which two fluids from the plurality of the fluids are positioned; and
a particulate substrate to pass through the density-differential interface and the capillary force-supported interface.

10. The biological component processing system of claim 9, wherein the particulate substrate includes magnetizing particles, and wherein the vertically layered fluid column is spatially positioned adjacent to a magnet to provide a magnetic field.

11. The biological component processing system of claim 9, wherein the particulate substrate includes microparticles that have a density greater than the three fluids positioned in fluid layers, wherein the density of the microparticles is sufficient to allow the microparticles to pass through the density-differential interface and the capillary force-supported interface either by the force of gravity or centrifugation.

12. The biological component processing system of claim 9, wherein the particulate substrate includes particle surfaces that are associated with a biological component.

13. The biological component processing system of claim 9, further comprising a biological sample, wherein the particulate substrate includes particle surfaces that are surface-activated to bind with a biological component relative to secondary components in the biological fluid sample.

14. A method of processing a biological component from a biological sample, comprising:
loading a biological sample into a vertically layered fluid column including:
a plurality of fluids positioned in fluid layers,
a density-differential interface along which two fluids from the plurality of fluids are positioned, and
a capillary force-supported interface along which two fluids from the plurality of the fluids are positioned; and
passing a biological component of the biological sample through the density-differential interface and the capillary force-supported interface.

15. The method of claim 14, wherein the biological component is associated with a particulate substrate that is passed through the density-differential interface and the capillary force-supported interface via a force selected from gravity, a centrifugal force, a magnetic field, buoyance, or a combination thereof.
